(19) United States
(12) Patent Application Publication
(10) Pub. No.: US 2024/0383822 A1
(43) Pub. Date: Nov. 21, 2024

(54) A PROCESS FOR PREPARING AT LEAST ONE AROMATIC ISOCYANATE

(71) Applicant: BASF SE, Ludwigshafen am Rhein (DE)

(72) Inventors: Jonas MATTERN, Schwarzheide (DE); Johannes BUETTNER, Schwarzheide (DE); Ruediger FRITZ, Bernsdorf (DE); Maximilian KOELLER, Schwarzheide (DE); Jan Pablo JOSCH, Ludwigshafen am Rhein (DE)

(21) Appl. No.: 18/692,911

(22) PCT Filed: Sep. 19, 2022
(86) PCT No.: PCT/EP2022/075965

§ 371 Date: Mar. 18, 2024

(30) Foreign Application Priority Data
Sep. 20, 2021 (EP) 21197762.4

(51) Int. Cl. C07B 43/10; B01J 19/24

(52) U.S. Cl. CPC C07B 43/10; B01J 19/245

(57) ABSTRACT

A chemical process for preparing at least one aromatic isocyanate, said process comprising n chemical sub-processes sp(i), wherein a sub-process sp(i) is carried out in a chemical processing sub-unit U(i), i=1 . . . n, n≥1, wherein during a regular operating mode of a sub-unit U(i), the sub-process sp(i) being carried out in said sub-unit U(i) comprises feeding an educt composition E(i) comprising at least one aromatic compound Z^{j}_{E}(i), j≥(i), into said sub-unit U(i), processing said composition E(i) in said sub-unit U(i) and obtaining a product composition P(i) comprising at least one aromatic compound Z^{k}ₚ(i), k≥1, wherein the chemical process comprises starting up said sub-process sp(i) in said sub-unit U(i), wherein said starting-up comprises processing a chemical start-up composition S(i) in said sub-unit U(i), wherein said S(i) #E(i) and wherein said S(i) comprises at least one of the aromatic compounds Z^{k}ₚ(i).

The present invention relates to a process for preparing at least one aromatic isocyanate comprising a specific starting-up, and to a chemical unit U for carrying out said process.

Aromatic isocyanates are often prepared via a continuous or discontinuous process in suitable plant sections or plants. Since such a process comprises several sub-processes, a plant section or plant comprises one or more chemical processing sub-units for processing the sub-processes. It is also possible to carry out a sub-process in parallel in several chemical processing sub-units. Considering that preparing an aromatic isocyanate usually includes nitration, hydrogenation and phosgenation of an aromatic compound, as for example in the preparation of toluene diisocyanate starting from toluene, a process for preparing at least one aromatic isocyanate includes several sub-processes wherein a sub-process is carried out in a chemical processing sub-unit. The product compositions obtained from the chemical processing units are then fed to further processing in subsequent chemical processing sub-units until a desired product composition is obtained.

During a regular operating mode of a sub-unit, the sub-process being carried out in said sub-unit thus comprises feeding an educt composition comprising at least one aromatic compound into said sub-unit and processing said composition in said sub-unit for obtaining a product composition comprising at least one aromatic compound, such that eventually at least one aromatic isocyanate is obtained from the latest chemical processing sub-unit. As used herein, an educt composition is equivalent to a reactant composition. Likewise the term educt, also spelled Edukt in the German language means reactant as defined by Baerns, M. et al in “Technische Chemie”, Wiley, 2014, page 697. Therefore, as defined in the present invention, an educt composition is equivalent to a reactant composition.

In order to ensure a high performance of the one or more chemical processing units and chemical processing sub-units of a plant section or plant, regular maintenance, cleaning and optionally repairing of said chemical processing units or chemical processing sub-units is required.

For doing so, plant sections or plants comprising one or more chemical processing units and chemical processing sub-units have to be shut down in parts or completely. Parts of a plant section or plant not affected by maintenance, cleaning or repairing can stay in steady state, in particular if a return of a stream comprising a product composition or another circulating stream can be ensured. For instance, WO 2015/197522 A1 discloses a method in which plant sections not affected by maintenance, cleaning or repairing are operated in a so-called recirculation mode. It is disclosed therein that the product of such a plant section or of a further downstream plant section is fed into this plant section to maintain a circulating flow. In WO 2017/050776 A1 a similar method is disclosed in which plant sections not affected by maintenance, cleaning or repairing are operated under a circulating flow.

On the other hand, those parts of a plant section or plant which have been affected by maintenance have to be re-started. However, starting-up of a process carried out in one or more chemical processing units and chemical processing sub-units is an expensive procedure, in particular considering resource efficiency and energy costs. Resource efficiency relates to the influence of process parameters different to target process parameters on process performance. If for example an educt composition differs from a target educt composition having an optimized composition for further processing, an inferior conversion may be expected making recycling or wasting of excess starting material necessary. Energy costs include costs for heating or cooling of chemical processing units, chemical processing sub-units, separation devices, and other processing devices. Additionally, starting-up of a process can be time consuming. In the context of the present invention starting-up of a process is terminated in particular when the product composition meets the desired specifications, inter alia allowing further processing thereof in a subsequent process or process step.

Therefore, it was an object of the present invention to provide a novel process for preparing at least one aromatic isocyanate comprising a specific starting-up process. In particular, it was an object of the present invention to provide an improved process for preparing at least one aromatic isocyanate comprising a specific starting-up concerning resource efficiency.

Therefore, the present invention relates to a process for preparing at least one aromatic isocyanate, said process comprising n chemical sub-processes sp(i), wherein a sub-process sp(i) is carried out in a chemical processing sub-unit U(i), i=1 . . . n, n≥1, wherein during a regular operating mode of a sub-unit U(i), the sub-process sp(i) being carried out in said sub-unit U(i) comprises feeding an educt composition E(i) comprising at least one aromatic compound Z^{j}_{E}(i), j≥1, into said sub-unit U(i), processing said composition E(i) in said sub-unit U(i) and obtaining a product composition P(i) comprising at least one aromatic compound Z^{k}ₚ(i), k≥1,

wherein the chemical process comprises starting up said sub-process sp(i) in said sub-unit U(i), wherein said starting-up comprises processing a chemical start-up composition S(i) in said sub-unit U(i), wherein said S(i)≠E(i) and wherein said S(i) comprises at least one of the aromatic compounds Z^{k}_{P}(i).

As mentioned above plant sections or plants comprising one or more chemical processing units and chemical processing sub-units have to be shut down in parts or completely if maintenance, cleaning or repairing needs to be carried out. Thus, it is preferred that the process according to the present invention further comprises, prior to starting up the sub-process sp(i) in the sub-unit U(i), shutting down the sub-process sp(i) in the sub-unit U(i) and keeping the sub-unit U(i) in a non-operation mode for a period of time Δt(i).

For starting up the sub-process sp(i) of the process according to the present invention, it is preferred that said starting up comprises feeding the chemical composition S(i) into the sub-unit U(i).

As defined above, the process according to the present invention comprises n chemical sub-processes sp(i), wherein a sub-process sp(i) is carried out in a chemical processing sub-unit U(i), i=1 . . . n, n≥1. In the case where n=1 the process comprises only one sub-process being carried out in a single sub-unit which can be considered as a chemical unit U. In the case where n≥2, at least 2 sub-processes are comprised by the process of the present invention each being carried out in a chemical processing sub-unit. If n≥2, thus it n is at least 2, it is preferred that all sub-units U(i) are part of a chemical unit U.

In the case where all sub-units U(i) are part of a chemical unit U, it is preferred that U further comprises, in addition to the n chemical processing sub-units U(i), r storing devices D(m), m=1 . . . r, r≥1. If the chemical unit U further comprises, in addition to the n chemical processing sub-units U(i), r storing devices D(m), m=1 . . . r, r≥1, it is preferred that the process according to the present invention further comprises, during a regular operating mode of a sub-unit U(i), removing the product composition P(i) from said sub-unit U(i) and transferring a portion of said product composition P(i) removed from said sub-unit U(i) into a storing device D(m).

In the case where the product composition P(i) is removed from a sub-unit U(i) during a regular operating mode of said sub-unit U(i) and where a portion of said product composition P(i) removed from said sub-unit U(i) is transferred into a storing device D(m), it is preferred that, during starting up the sub-process sp(i), at least a portion of P(i), stored in D(m) during the regular operating mode of the sub-unit U(i), is fed as at least a portion of the chemical composition S(i) into the sub-unit U(i).

According to the present invention a chemical sub-process sp(i) is carried out in a chemical processing sub-unit U(i). Thus, a chemical processing sub-unit U(i) can be any conceivable means suitable for carrying out a chemical sub-process sp(i). It is preferred that the sub-unit U(i) is selected from the group consisting of a chemical reactor, a distillation column, a rectification column, a tank, a phase separator, a washing device, and a combination of two or more thereof.

According to the present invention the process for preparing at least one aromatic isocyanate comprises n chemical sub-processes sp(i). It is preferred that at least one sub-process sp(i) comprises a nitration, a hydrogenation, a phosgenation, washing, a phase separation, or a distillation.

In the case where a sub-process sp(i) comprises a nitration, this can be indicated by an index N. Thus, a sub-process comprising a nitration can be designated as sp_{N}(i). Similarly, a sub-process comprising a hydrogenation can be designated as sp_{H}(i), a sub-process comprising a phosgenation as sp_{P}(i), a sub-process comprising a washing as sp_{W}(i), a sub-process comprising a separation as sp_{S}(i), and a sub-process comprising a distillation as sp_{D}(i). The same applies to a sub-unit U(i), an educt composition E(i), a storing device D(m), a product composition P(i) or a chemical start-up composition relating to a sub-process as defined above.

It is preferred that at least one sub-process sp(i) comprises a nitration and at least one further sub-process sp(i) comprises a hydrogenation. Also, it is preferred that at least one further sub-process sp(i) comprises a phosgenation. Thus, it is particularly preferred that at least one sub-process sp(i) comprises a nitration, that at least one further sub-process sp(i) comprises a hydrogenation, and that at least one further sub-process sp(i) comprises a phosgenation.

It is preferred that the at least one aromatic isocyanate comprises an aromatic diisocyanate, preferably a toluene diisocyanate, more preferably one or more of 2,4-toluene diisocyanate and 2,6-toluene diisocyanate, or a methylene diphenyl diisocyanate, preferably one or more of methylene diphenyl 2,2′-diisocyanate, methylene diphenyl 2,4′-diisocyanate, and methylene diphenyl 4,4′-diisocyanate.

It is particularly preferred that at least one sub-process sp(i) comprises a nitration, a hydrogenation, or a phosgenation, more preferably at least one sub-process sp(i) comprises a nitration, at least one further sub-process sp(i) comprises a hydrogenation, and at least one further sub-process sp(i) comprises a phosgenation, wherein the at least one aromatic isocyanate comprises an aromatic diisocyanate, preferably a toluene diisocyanate, more preferably one or more of 2,4-toluene diisocyanate and 2,6-toluene diisocyanate, or a methylene diphenyl diisocyanate, preferably one or more of methylene diphenyl 2,2′-diisocyanate, methylene diphenyl 2,4′-diisocyanate, and methylene diphenyl 4,4′-diisocyanate. Further, it is particularly preferred that the at least one aromatic isocyanate comprises, preferably is, one or more of 2,4-toluene diisocyanate and 2,6-toluene diisocyanate.

In accordance with the above, it is preferred that one or more sub-processes sp(i) comprise a nitration, sp_{N}(i). In accordance with the above, a sub-unit U(i) in which a sub-process sp_{N}(i) is carried out is also designated herein as U_{N}(i), an educt composition relating to a sub-process sp_{N}(i) is designated herein as E_{N}(i), and a product composition P(i) obtained from a sub-unit U_{N}(i) is designated herein as P_{N}(i). Thus, it is preferred that one or more sub-processes sp(i) comprise a nitration, sp_{N}(i), wherein each sub-unit U_{N}(i) in which a sub-process sp_{N}(i) is carried out comprises a nitration reactor, wherein an educt composition E_{N}(i), being fed into a sub-unit U_{N}(i), comprises at least one nitrating agent, preferably nitric acid, and preferably further comprises at least one water scavenger, preferably sulfuric acid.

More preferably, the process according to the present invention comprises two or more sub-processes sp_{N}(i) which are carried out in sequence, wherein at last a portion of the product composition P_{N}(i) obtained from a sub-unit U_{N}(i) is fed, optionally after an intermediate treatment sub-process, as educt composition E_{N}(i+1) into the sub-unit U_{N}(i+1) downstream of U_{N}(i) during a regular operating mode of U_{N}(i+1), wherein the educt composition E_{N}(1) being fed into the first, most upstream sub-unit U_{N}(1) comprises toluene as Z¹_{E,N}(1), wherein more preferably, j=1.

Further, it is preferred that during a regular operating mode of a sub-unit U_{N}(i), a portion of P_{N}(i) is stored in at least one storing device D_{N}(m). As defined hereinabove, r storing devices D(m), m=1 . . . r, r≥1, can be comprised in the chemical unit U in addition to the n chemical processing sub-units U(i). Thus, it is particularly preferred that that the process according to the present invention further comprises, during a regular operating mode of a sub-unit U_{N}(i), removing the product composition P_{N}(i) from said sub-unit U_{N}(i) and transferring a portion of said product composition P_{N}(i) removed from said sub-unit U_{N}(i) into a storing device D_{N}(m).

In the case where the process according to the present invention further comprises, during a regular operating mode of a sub-unit U_{N}(i), removing the product composition P_{N}(i) from said sub-unit U_{N}(i) and transferring a portion of said product composition P_{N}(i) removed from said sub-unit U_{N}(i) into a storing device D_{N}(m), it is preferred that said process comprises starting up a sub-process sp_{N}(i) in a sub-unit U_{N}(i), wherein said starting up comprises feeding at least a portion of P_{N}(i), obtained from said U_{N}(i) during regular operation mode of U_{N}(i), and stored in the at least one storing device D_{N}(m), as S_{N}(i) into U_{N}(i).

In the case where one or more sub-processes sp(i) comprise a nitration, sp_{N}(i), wherein each sub-unit U_{N}(i) in which a sub-process sp_{N}(i) is carried out comprises a nitration reactor, wherein an educt composition E_{N}(i), being fed into a sub-unit U_{N}(i), comprises at least one nitrating agent, it is preferred that a product composition P_{N}(i), obtained from a sub-unit U_{N}(i), comprises one or more of 2,4-dinitrotoluene as Z¹_{P,N}(i), 2,6-dinitrotoluene as Z²_{P,N}(i), and 2-nitrotoluene as Z³_{P,N}(i).

Further in the case where one or more sub-processes sp(i) comprise a nitration, sp_{N}(i), wherein each sub-unit U_{N}(i) in which a sub-process sp_{N}(i) is carried out comprises a nitration reactor, wherein an educt composition E_{N}(i), being fed into a sub-unit U_{N}(i), comprises at least one nitrating agent, it is preferred that the process further comprises at least one first intermediate treatment sub-process, wherein a first intermediate treatment sub-process comprises, preferably consists of, a phase separation sub-process sp_{S}(i). Said phase separation sub-process sp_{S}(i) is carried out in a sub-unit which is also designated herein as U_{S}(i), wherein the product composition P_{N}(i) obtained from U_{N}(i), prior to being fed to the sub-unit U_{N}(i+1), is subjected as educt composition E_{S}(i) to separation and is separated during sp_{S}(i) in an organic phase and an aqueous phase, wherein the organic phase, which is also designated herein as P_{S}(i), is fed as educt composition E_{N}(i+1) into the sub-unit U_{N}(i+1), wherein the sub-unit U_{S}(i) in which sp_{S}(i) is carried out comprises a phase separator.

If the process according to the present invention comprises at least one first intermediate treatment sub-process as defined above, it is preferred that a portion of P_{S}(i) is stored in at least one storing device D_{S}(m) during a regular operating mode of a sub-unit U_{S}(i). As defined hereinabove, r storing devices D(m), m=1 . . . r, r≥1, can be comprised in the chemical unit U in addition to the n chemical processing sub-units U(i). Thus, it is particularly preferred that the process according to the present invention further comprises, during a regular operating mode of a sub-unit U_{S}(i), removing the product composition P_{S}(i) from said sub-unit U_{S}(i) and transferring a portion of said product composition P_{S}(i) removed from said sub-unit U_{S}(i) into a storing device D_{S}(m).

In the case where the process of the present invention comprises storing a portion of P_{S}(i) in at least one storing device D_{S}(m) during a regular operating mode of a sub-unit U_{S}(i), it is preferred that the process comprises starting up the sub-process sp_{S}(i) in the sub-unit U_{S}(i), wherein said starting up comprises feeding at least a portion of P_{S}(i), obtained from said U_{S}(i) during a regular operating mode of U_{S}(i) and stored in the at least one storing device D_{S}(m), as chemical start-up composition S_{S}(i) into U_{S}(i).

Further in the case where the process of the present invention comprises storing a portion of P_{S}(i) in at least one storing device D_{S}(m) during a regular operating mode of a sub-unit U_{S}(i), it is preferred that the process comprises starting up the sub-process sp_{N}(i) in a sub-unit U_{N}(i), wherein said starting up comprises feeding at least a portion of P_{S}(i), obtained from said U_{S}(i) and stored in the at least one storing device D_{N}(m), as chemical start-up composition S_{N}(i) into U_{N}(i).

In the case where the process further comprises at least one first intermediate treatment sub-process, wherein a first intermediate treatment sub-process comprises, preferably consists of, a phase separation sub-process sp_{S}(i) as defined above, it is preferred that a product composition P_{S}(i), obtained from a sub-unit U_{S}(i), comprises one or more of 2,4-dinitrotoluene as Z¹_{P,S}(i), 2,6-dinitrotoluene as Z²_{P,S}(i), and 2-nitrotoluene as Z³_{P,S}(i).

Further in the case where the process further comprises at least one first intermediate treatment sub-process, wherein a first intermediate treatment sub-process comprises, preferably consists of, a phase separation sub-process sp_{S}(i) as defined above, it is preferred that the product composition P_{S}(i), obtained from the most downstream sub-unit U_{S}(i), comprises one or more of 2,4-dinitrotoluene as Z¹_{P},s(i) and 2,6-dinitrotoluene as Z²_{P,S}(i), wherein at least 75 mol-%, preferably at least 80 mol-%, more preferably at least 85 mol-%, more preferably at least 90 mol-% of all Z^{k}_{P,S}(i) comprised in the product composition P_{S}(i) obtained from the most downstream sub-unit U_{S}(i) consist of one or more of 2,4-dinitrotoluene as Z¹_{P,S}(i) and 2,6-dinitrotoluene as Z²_{P,S}(i).

As noted above, it is preferred that one or more sub-processes sp(i) comprise a nitration, sp_{N}(i), wherein each sub-unit U_{N}(i) in which a sub-process sp_{N}(i) is carried out comprises a nitration reactor. In this connection, it is further preferred that an educt composition E_{N}(i), being fed into a sub-unit U_{N}(i), comprises at least one nitrating agent and preferably further comprises at least one water scavenger. In such a case, it is preferred that the process further comprises at least one second intermediate treatment sub-process, wherein a second intermediate treatment sub-process comprises, preferably consists of a washing sub-process, sp_{W}(i), which is carried out in a sub-unit U_{W}(i), wherein a product composition P_{N}(i) obtained from a sub-unit U_{N}(i), or a product composition P_{S}(i) obtained from a sub-unit U_{S}(i), is subjected as educt composition E_{W}(i) to washing, obtaining a product composition P_{W}(i), wherein the sub-unit U_{W}(i) in which sp_{W}(i) is carried out comprises a washing device.

In the case where the process further comprises at least one second intermediate treatment sub-process as defined above, it is preferred that the process comprises one sub-process sp_{W}(1), wherein sp_{W}(1) is carried out directly downstream of the most downstream sub-process sp(i), or directly downstream of the most downstream sub-process sp_{S}(i), or directly downstream of the most downstream sub-process sp_{N}(i) and directly downstream of the most downstream sub-process sp_{S}(i), preferably directly downstream of the most downstream sub-process sp_{S}(i).

Further in the case where the process further comprises at least one second intermediate treatment sub-process as defined above, it is preferred that the process comprises, during a regular operating mode of a sub-unit U_{W}(i), storing a portion of P_{W}(i) in at least one storing device D_{W}(m). As defined hereinabove, r storing devices D(m), m=1 . . . r, r≥1, can be comprised in the chemical unit U in addition to the n chemical processing sub-units U(i).

If a portion of P_{W}(i) is stored in at least one storing device D_{W}(m) during a regular operating mode of a sub-unit U_{W}(i), it is preferred that the process comprises starting up a sub-process sp_{W}(i) in a sub-unit U_{W}(i), wherein said starting up comprises feeding at least a portion of P_{W}(i), obtained from said U_{W}(i) and stored in the at least one storing device D_{W}(m), as S_{W}(i) into U_{W}(i).

Further in the case where the process further comprises at least one second intermediate treatment sub-process as defined above, it is preferred that a product composition P_{W}(i), obtained from a sub-unit U_{W}(i), comprises one or more of 2,4-dinitrotoluene as Z¹_{P,W}(i), 2,6-dinitrotoluene as Z²_{P,W}(i), and 2-nitrotoluene as Z³_{P,W}(i).

Further in the case where the process further comprises at least one second intermediate treatment sub-process as defined above, it is preferred that the product composition P_{W}(i), obtained from the most downstream sub-unit U_{W}(i), comprises one or more of 2,4-dinitrotoluene as Z¹_{P,W}(i) and 2,6-dinitrotoluene as Z²_{P,W}(i), wherein at least 75 mol-%, preferably at least 80 mol-%, more preferably at least 85 mol-%, more preferably at least 90 mol-% of all Z^{k}_{P,W}(i) comprised in the product composition P_{W}(i) obtained from the most downstream sub-unit U_{W}(i) consist of one or more of 2,4-dinitrotoluene as Z¹_{P,W}(i) and 2,6-dinitrotoluene as Z²_{P,W}(i).

It is preferred that one or more sub-processes sp(i) comprise a hydrogenation, sp_{H}(i), wherein each sub-unit U_{H}(i) in which a sub-process sp_{H}(i) is carried out comprises a hydrogenation reactor, wherein an educt composition E_{H}(i), being fed into a sub-unit U_{H}(i), comprises at least one hydrogenation agent.

It is further preferred that one or more sub-processes sp(i) comprise a nitration, sp_{N}(i), as defined herein, and that one or more sub-processes sp(i) comprise a hydrogenation, sp_{H}(i), wherein each sub-unit U_{H}(i) in which a sub-process sp_{H}(i) is carried out comprises a hydrogenation reactor, wherein an educt composition E_{H}(i), being fed into a sub-unit U_{H}(i), comprises at least one hydrogenation agent.

It is particularly preferred that one or more sub-processes sp(i) comprise a nitration, sp_{N}(i), as defined herein, that the process comprises at least one first intermediate treatment sub-process as defined herein, and that one or more sub-processes sp(i) comprise a hydrogenation, sp_{H}(i), wherein each sub-unit U_{H}(i) in which a sub-process sp_{S}(i) is carried out comprises a hydrogenation reactor, wherein an educt composition E_{H}(i), being fed into a sub-unit U_{H}(i), comprises at least one hydrogenation agent.

It is more particularly preferred that one or more sub-processes sp(i) comprise a nitration, sp_{N}(i), as defined herein, that the process comprises at least one first intermediate treatment sub-process as defined herein, that the process comprises at least one second intermediate treatment sub-process as defined herein, and that one or more sub-processes sp(i) comprise a hydrogenation, sp_{H}(i), wherein each sub-unit U_{H}(i) in which a sub-process sp_{H}(i) is carried out comprises a hydrogenation reactor, wherein an educt composition E_{H}(i), being fed into a sub-unit U_{H}(i), comprises at least one hydrogenation agent.

In the case where one or more sub-processes sp(i) comprise a hydrogenation, sp_{H}(i), as defined herein, it is preferred that the process comprises one sub-process sp_{H}(1).

Further in the case where one or more sub-processes sp(i) comprise a hydrogenation, sp_{H}(i), as defined herein, it is preferred that the at least one aromatic compound Z^{j}_{E,H}(i) comprised in E_{H}(i) is one or more of 2,4-dinitrotoluene as Z¹_{E,H}(i), 2,6-dinitrotoluene as Z²_{E,H}(i), 2-nitrotoluene as Z³_{E,H}(i) and 6-nitrotoluene as Z⁴_{E,H}(i).

Further in the case where one or more sub-processes sp(i) comprise a hydrogenation, sp_{H}(i), as defined herein, it is preferred that E_{H}(i) comprises one or more of a product composition P_{N}(i) as defined in any one of the embodiments disclosed herein, a product composition P_{S}(i) as defined in any one of the embodiments disclosed herein, and a product composition P_{W}(i) as defined in any one of the embodiments disclosed herein, preferably a product composition P_{W}(i) obtained from washing as defined in any one of the embodiments disclosed herein, preferably a product composition P_{W}(i) obtained from the most downstream sub-unit U_{W}(i), and comprising one or more of 2,4-dinitrotoluene as Z¹_{P,W}(i) and 2,6-dinitrotoluene as Z²_{P,W}(i), wherein at least 75 mol-%, preferably at least 80 mol-%, more preferably at least 85 mol-%, more preferably at least 90 mol-% of all Z^{k}_{P,W}(i) comprised in the product composition P_{W}(i) obtained from the most downstream sub-unit U_{W}(i) consist of one or more of 2,4-dinitrotoluene as Z¹_{P,W}(i) and 2,6-dinitrotoluene as Z²_{P,W}(i).

Further in the case where one or more sub-processes sp(i) comprise a hydrogenation, sp_{H}(i), as defined herein, it is preferred that the process comprises a portion of P_{H}(i) is stored in at least one storing device D_{H}(m) during a regular operating mode of a sub-unit U_{H}(i). As defined hereinabove, r storing devices D(m), m=1 . . . r, r≥1, can be comprised in the chemical unit U in addition to the n chemical processing sub-units U(i).

If a portion of P_{H}(i) is stored in at least one storing device D_{H}(m) during a regular operating mode of a sub-unit U_{H}(i), it is preferred that the process comprises starting up a sub-process sp_{H}(i) in a sub-unit U_{H}(i), wherein said starting up comprises feeding at least a portion of P_{H}(i), obtained from said U_{H}(i) and stored in the at least one storing device D_{H}(m), as S_{H}(i) into U_{H}(i). Further in the case where one or more sub-processes sp(i) comprise a hydrogenation, sp_{H}(i), as defined herein, it is preferred that a product composition P_{H}(i), obtained from a sub-unit U_{H}(i), comprises one or more of 2,4-diaminotoluene as Z¹_{P,H}(i), 2,6-diaminotoluene as Z²_{P,H}(i), and 2-aminotoluene as Z³_{P,H}(i).

Further in the case where one or more sub-processes sp(i) comprise a hydrogenation, sp_{H}(i), as defined herein, it is preferred that the process comprises starting up a sub-process sp_{H}(i) in a sub-unit U_{H}(i), said starting up comprising feeding at least a portion of P_{D}(i), obtained from U_{D}(i) and stored in at least one storing device D_{D,P}(m) as defined hereinbelow, as S_{H}(i) into U_{H}(i).

Further in the case where one or more sub-processes sp(i) comprise a hydrogenation, sp_{H}(i), as defined herein, it is preferred that the process comprises starting up a sub-process sp_{H}(i) in a sub-unit U_{H}(i), wherein said starting up comprises feeding at least a portion of U_{D}(i), obtained from U_{D}(i) and stored in at least one storing device D_{D,C}(m) as defined hereinbelow, as S_{H}(i) into U_{H}(i).

Further in the case where one or more sub-processes sp(i) comprise a hydrogenation, sp_{H}(i), as defined herein, it is preferred that the process further comprises at least one third intermediate treatment sub-process, wherein a third intermediate treatment sub-process comprises, preferably consists of, a distillation sub-process, sp_{D}(i), which is carried out in a sub-unit U_{D}(i), wherein preferably, a product composition P_{H}(i) obtained from a sub-unit U_{H}(i) is subjected as educt composition E_{D}(i) to distillation, obtaining a product composition P_{D}(i), wherein the sub-unit U_{D}(i) in which sp_{D}(i) is carried out comprises a distillation device and wherein, in U_{D}(i), E_{D}(i) is separated in P_{D}(i) and at least one further distillation composition C_{D}(i).

If the process further comprises at least one third intermediate treatment sub-process as defined above, it is preferred that a portion of P_{D}(i) is stored in at least one storing device D_{D,P}(m) during a regular operating mode of a sub-unit U_{D}(i). As defined hereinabove, r storing devices D(m), m=1 . . . r, r≥1, can be comprised in the chemical unit U in addition to the n chemical processing sub-units U(i).

In the case where a portion of P_{D}(i) is stored in at least one storing device D_{D,P}(m) during a regular operating mode of a sub-unit U_{D}(i), it is preferred that the process comprises starting up a sub-process sp_{D}(i) in a sub-unit U_{D}(i), wherein said starting up comprises feeding at least a portion of P_{D}(i), obtained from said U_{D}(i) and stored in the at least one storing device D_{D,P}(m), as S_{D}(i) into U_{D}(i).

If the process comprises starting up a sub-process sp_{H}(i) in a sub-unit U_{H}(i), wherein said starting up comprises feeding at least a portion of P_{D}(i), obtained from U_{D}(i) and stored in at least one storing device D_{D,P}(m) as defined hereinabove, as S_{H}(i) into U_{H}(i), it is preferred that a portion of C_{D}(i) is stored in at least one storing device D_{D,C}(m) during a regular operating mode of a sub-unit U_{D}(i). As defined hereinabove, r storing devices D(m), m=1 . . . r, r≥1, can be comprised in the chemical unit U in addition to the n chemical processing sub-units U(i).

In the case where a portion of C_{D}(i) is stored in at least one storing device D_{D,C}(m) during a regular operating mode of a sub-unit U_{D}(i), it is preferred that the process comprises starting up a sub-process sp_{D}(i) in a sub-unit U_{D}(i), wherein said starting up comprises feeding at least a portion of C_{D}(i), obtained from said U_{D}(i) and stored in the at least one storing device D_{D,C}(m), as S_{D}(i) into U_{D}(i).

If the process comprises starting up a sub-process sp_{H}(i) in a sub-unit U_{H}(i), wherein said starting up comprises feeding at least a portion of P_{D}(i), obtained from U_{D}(i) and stored in at least one storing device D_{D,P}(m) as defined hereinabove, as S_{H}(i) into U_{H}(i), it is preferred that a product composition P_{D}(i), obtained from a sub-unit U_{D}(i), comprises one or more of 2,4-diaminotoluene as Z¹_{P,D}(i), 2,6-diaminotoluene as Z²_{P,D}(i), and 2-aminotoluene as Z³_{P,D}(i).

It is preferred that the weight ratio of the total weight of 2,4-diaminotoluene as Z¹_{P,D}(i), 2,6-diaminotoluene as Z²_{P,D}(i), and 2-aminotoluene as Z³_{P,D}(i) comprised in P_{D}(i) relative to the total weight of 2,4-diaminotoluene as Z¹_{P,H}(i), 2,6-diaminotoluene as Z²_{P,H}(i), and 2-aminotoluene as Z³_{P,H}(i) comprised in P_{H}(i) is greater than 1:1, more preferably in the range of from 1.05:1 to 2.5:1, more preferably in the range of from 1.3:1 to 2.2:1, more preferably in the range of from 1.5:1 to 2.1:1.

It is preferred that the weight ratio of the product composition P_{D}(i) relative to the at least one further distillation composition C_{D}(i) is in the range of from 25:1 to 2:1, more preferably in the range of from 20:1 to 5:1.

Further in the case where the process comprises starting up a sub-process sp_{H}(i) in a sub-unit U_{H}(i), wherein said starting up comprises feeding at least a portion of P_{D}(i), obtained from U_{D}(i) and stored in at least one storing device D_{D,P}(m) as defined hereinabove, as S_{H}(i) into U_{H}(i), it is preferred that the process comprises starting up a sub-process sp_{D}(i) in a sub-unit U_{D}(i), wherein said starting up comprises feeding at least a portion of P_{H}(i), obtained from U_{H}(i) and stored in the at least one storing device D_{H}(m) as defined herein, as S_{D}(i) into U_{D}(i).

As defined above, it is preferred that one or more sub-processes sp(i) comprise a nitration, sp_{N}(i), wherein each sub-unit U_{N}(i) in which a sub-process sp_{N}(i) is carried out comprises a nitration reactor, wherein an educt composition E_{N}(i), being fed into a sub-unit U_{N}(i), comprises at least one nitrating agent, preferably nitric acid, and preferably further comprises at least one water scavenger, preferably sulfuric acid. In such a case, it is preferred that one or more sub-processes sp(i) comprise a phosgenation, sp_{P}(i), wherein each sub-unit U_{P}(i) in which a sub-process sp_{P}(i) is carried out comprises a phosgenation reactor, wherein an educt composition E_{P}(i), being fed into a sub-unit U_{P}(i), comprises at least one phosgenation agent.

As defined herein, it is preferred that one or more sub-processes sp(i) comprise a nitration, sp_{N}(i). In such a case, it is preferred that one or more sub-processes sp(i) comprise a phosgenation, sp_{P}(i), wherein each sub-unit U_{P}(i) in which a sub-process sp_{P}(i) is carried out comprises a phosgenation reactor, wherein an educt composition E_{P}(i), being fed into a sub-unit U_{P}(i), comprises at least one phosgenation agent.

It is further preferred that one or more sub-processes sp(i) comprise a nitration, sp_{N}(i), as defined herein, that the process comprises at least one first intermediate treatment sub-process as defined herein, and that one or more sub-processes sp(i) comprise a phosgenation, sp_{P}(i), wherein each sub-unit U_{P}(i) in which a sub-process sp_{P}(i) is carried out comprises a phosgenation reactor, wherein an educt composition E_{P}(i), being fed into a sub-unit U_{P}(i), comprises at least one phosgenation agent.

It is further preferred that one or more sub-processes sp(i) comprise a nitration, sp_{N}(i), as defined herein, that the process comprises at least one first intermediate treatment sub-process as defined herein, that the process comprises at least one second intermediate treatment sub-process as defined herein, and that one or more sub-processes sp(i) comprise a phosgenation, sp_{P}(i), wherein each sub-unit U_{P}(i) in which a sub-process sp_{P}(i) is carried out comprises a phosgenation reactor, wherein an educt composition E_{P}(i), being fed into a sub-unit U_{P}(i), comprises at least one phosgenation agent.

It is further preferred that one or more sub-processes sp(i) comprise a nitration, sp_{N}(i), as defined herein, that the process comprises at least one first intermediate treatment sub-process as defined herein, that the process comprises at least one second intermediate treatment sub-process as defined herein, that one or more sub-processes sp(i) comprise a hydrogenation, sp_{H}(i), as defined herein, and that one or more sub-processes sp(i) comprise a phosgenation, sp_{P}(i), wherein each sub-unit U_{P}(i) in which a sub-process sp_{P}(i) is carried out comprises a phosgenation reactor, wherein an educt composition E_{P}(i), being fed into a sub-unit U_{P}(i), comprises at least one phosgenation agent.

It is further preferred that one or more sub-processes sp(i) comprise a nitration, sp_{N}(i), as defined herein, that the process comprises at least one first intermediate treatment sub-process as defined herein, that the process comprises at least one second intermediate treatment sub-process as defined herein, that one or more sub-processes sp(i) comprise a hydrogenation, sp_{H}(i), as defined herein, that the process further comprises at least one third intermediate treatment sub-process as defined herein, and that one or more sub-processes sp(i) comprise a phosgenation, sp_{P}(i), wherein each sub-unit U_{P}(i) in which a sub-process sp_{P}(i) is carried out comprises a phosgenation reactor, wherein an educt composition E_{P}(i), being fed into a sub-unit U_{P}(i), comprises at least one phosgenation agent.

If one or more sub-processes sp(i) comprise a phosgenation, sp_{P}(i), as defined herein, it is preferred that the process comprises one sub-process sp_{P}(1).

Further in the case where one or more sub-processes sp(i) comprise a phosgenation, sp_{P}(i), as defined herein, it is preferred that the at least one aromatic compound Z^{j}_{E.P}(i) comprised in E_{P}(i) is one or more of 2,4-diaminotoluene as Z¹_{E,P}(i), 2,6-diaminotoluene as Z²_{E,P}(i), 2-aminotoluene as Z³_{E,P}(i) and 6-aminotoluene as Z⁴_{E,P}(i).

Further in the case where one or more sub-processes sp(i) comprise a phosgenation, sp_{P}(i), as defined herein, it is preferred that E_{P}(i) comprises one or more of a product composition P_{H}(i) as defined in any one of the embodiments disclosed herein, a product composition P_{D}(i) as defined in any one of the embodiments disclosed herein, a product composition P_{D}(i) as defined in any one of the embodiments disclosed herein, a distillation composition C_{D}(i) as defined in any one of the embodiments disclosed herein, preferably a product composition P_{D}(i) obtained from a sub-unit U_{D}(i) as defined herein or a product composition P_{D}(i) obtained from the most downstream sub-unit U_{D}(i) as defined herein, more preferably a product composition P_{D}(i) obtained from the most downstream sub-unit U_{D}(i) as defined herein.

In the case where one or more sub-processes sp(i) comprise a phosgenation, sp_{P}(i), as defined herein, it is preferred that a portion of Pₚ(i) is stored in at least one storing device Dₚ(m) during a regular operating mode of a sub-unit U_{P}(i). As defined hereinabove, r storing devices D(m), m=1 . . . r, r≥1, can be comprised in the chemical unit U in addition to the n chemical processing sub-units U(i).

Further in the case where one or more sub-processes sp(i) comprise a phosgenation, sp_{P}(i), as defined herein, it is preferred that a portion of Pₚ(i) is stored in at least one storing device Dₚ(m) during a regular operating mode of a sub-unit U_{P}(i). As defined hereinabove, r storing devices D(m), m=1 . . . r, r≥1, can be comprised in the chemical unit U in addition to the n chemical processing sub-units U(i).

If a portion of Pₚ(i) is stored in at least one storing device Dₚ(m) during a regular operating mode of a sub-unit U_{P}(i), it is preferred that the process comprises starting up a sub-process sp_{P}(i) in a sub-unit U_{P}(i), wherein said starting up comprises feeding at least a portion of Pₚ(i), obtained from said U_{P}(i) and stored in the at least one storing device Dₚ(m), as Sₚ(i) into U_{P}(i).

Further in the case where one or more sub-processes sp(i) comprise a phosgenation, sp_{P}(i), as defined herein, it is preferred that a product composition Pₚ(i), obtained from a sub-unit U_{P}(i), comprises one or more of 2,4-toluene diisocyanate as Z¹_{P,P}(i), 2,6-toluene diisocyanate as Z²_{P,P}(i), and 2-toluene isocyanate as Z³_{P,P}(i).

It is preferred that the abovementioned process is a continuous process or discontinuous process, more preferably a continuous process.

Further, the present invention relates to a chemical unit U for carrying out the process according to any one of the embodiments defined herein.

It is preferred that the chemical unit comprises
(i.1) one or more sub-units U_{N}(i), preferably two sub-units U_{N}(1) and U_{N}(2);
(i.2) one or more sub-units U_{S}(i), preferably two sub-units U_{S}(1) and U_{S}(²);
(i.3) one or more sub-units U_{W}(i), preferably one sub-unit U_{W}(1);
(i.4) one or more storing devices D_{N}(m), preferably two storing devices D_{N}(1) and D_{N}(2);
   wherein U_{N}(1) is arranged upstream of U_{S}(1), U_{S}(1) is arranged upstream U_{N}(2), U_{N}(2) is arranged upstream U_{S}(2), and U_{S}(2) is arranged upstream of U_{W}(1);
(ii.1) one or more sub-units U_{H}(i), preferably one sub-unit U_{H}(1);
(ii.2) one or more sub-units U_{D}(i), preferably one sub-unit U_{D}(1);
(ii.3) one or more storing devices D_{H}(m), preferably one storing device D_{H}(1);
(ii.4) one or more storing devices D_{D}(m), preferably two storing devices D_{D,P}(1) and D_{D,C}(1);
   wherein U_{H}(1) is arranged downstream of U_{W}(1) and upstream U_{D}(1);
(iii.1) one or more sub-units U_{P}(i), preferably one sub-unit U_{P}(1);
   wherein U_{P}(1) is arranged downstream of U_{D}(1).

The present invention is further illustrated by the following set of embodiments and combinations of embodiments resulting from the dependencies and back-references as indicated. In particular, it is noted that in each instance where a range of embodiments is mentioned, for example in the context of a term such as “The chemical process of any one of embodiments 1 to 4”, every embodiment in this range is meant to be explicitly disclosed for the skilled person, i.e. the wording of this term is to be understood by the skilled person as being synonymous to “The chemical process of any one of embodiments 1, 2, 3 and 4”. Further, it is explicitly noted that the following set of embodiments is not the set of claims determining the extent of protection, but represents a suitably structured part of the description directed to general and preferred aspects of the present invention.
1. A chemical process for preparing at least one aromatic isocyanate, said process comprising n chemical sub-processes sp(i), wherein a sub-process sp(i) is carried out in a chemical processing sub-unit U(i), i=1 . . . n, n≥1, wherein during a regular operating mode of a sub-unit U(i), the sub-process sp(i) being carried out in said sub-unit U(i) comprises feeding an educt composition E(i) comprising at least one aromatic compound Z^{j}_{E}(i), j≥1, into said sub-unit U(i), processing said composition E(i) in said sub-unit U(i) and obtaining a product composition P(i) comprising at least one aromatic compound Z^{k}ₚ(i), k≥1, wherein the chemical process comprises starting up said sub-process sp(i) in said sub-unit U(i), wherein said starting-up comprises processing a chemical start-up composition S(i) in said sub-unit U(i), wherein said S(i)≠E(i) and wherein said S(i) comprises at least one of the aromatic compounds Z^{k}_{P}(i).
2. The process of embodiment 1, further comprising, prior to starting up the sub-process sp(i) in the sub-unit U(i), shutting down the sub-process sp(i) in the sub-unit U(i) and keeping the sub-unit U(i) in a non-operation mode for a period of time Δt(i).
3. The process of embodiment 1 or 2, wherein starting up the sub-process sp(i) comprises feeding the chemical composition S(i) into the sub-unit U(i).
4. The process of any one of embodiments 1 to 3, wherein, if n≥2, at least 2, preferably all sub-units U(i) are part of a chemical unit U.
5. The process of embodiment 4, wherein U further comprises, in addition to the n chemical processing sub-units U(i), r storing devices D(m), m=1 . . . r, r≥1.
6. The process of embodiment 5, further comprising, during a regular operating mode of a sub-unit U(i), removing the product composition P(i) from said sub-unit U(i) and transferring a portion of said product composition P(i) removed from said sub-unit U(i) into a storing device D(m).
7. The process of embodiment 6, further comprising, during starting up the sub-process sp(i), feeding at least a portion of P(i), stored in D(m) during the regular operating mode of the sub-unit U(i), as at least a portion of the chemical composition S(i) into the sub-unit U(i).
8. The process of any one of embodiments 1 to 7, wherein the sub-unit U(i) is selected from the group consisting of a chemical reactor, a distillation column, a rectification column, a tank, a phase separator, a washing device, and a combination of two or more thereof.
9. The process of any one of embodiments 1 to 8, wherein at least one sub-process sp(i) comprises a nitration and at least one further sub-process sp(i) comprises a hydrogenation.
10. The process of any one of embodiments 1 to 9, preferably of embodiment 9, wherein at least one further sub-process sp(i) comprises a phosgenation.
11. The process of any one of embodiments 1 to 10, preferably of any one of embodiments 8 to 10, wherein the at least one aromatic isocyanate comprises an aromatic diisocyanate, preferably a toluene diisocyanate, more preferably one or more of 2,4-toluene diisocyanate and 2,6-toluene diisocyanate, or a methylene diphenyl diisocyanate, preferably one or more of methylene diphenyl 2,2′-diisocyanate, methylene diphenyl 2,4′-diisocyanate, and methylene diphenyl 4,4′-diisocyanate.
12. The process of embodiment 11, wherein the at least one aromatic isocyanate comprises, preferably is, one or more of 2,4-toluene diisocyanate and 2,6-toluene diisocyanate.
13. The process of any one of embodiments 1 to 12, preferably of any one of embodiments 8 to 12, wherein one or more sub-processes sp(i) comprise a nitration, sp_{N}(i), wherein each sub-unit U_{N}(i) in which a sub-process sp_{N}(i) is carried out comprises a nitration reactor, wherein an educt composition E_{N}(i), being fed into a sub-unit U_{N}(i), comprises at least one nitrating agent, preferably nitric acid, and preferably further comprises at least one water scavenger, preferably sulfuric acid;
wherein said process preferably comprises two or more sub-processes sp_{N}(i) which are carried out in sequence, wherein at last a portion of the product composition P_{N}(i) obtained from a sub-unit U_{N}(i) is fed, optionally after an intermediate treatment sub-process, as educt composition E_{N}(i+1) into the sub-unit U_{N}(i+1) downstream of U_{N}(i) during a regular operating mode of U_{N}(i+1);
wherein the educt composition E_{N}(1) being fed into the first, most upstream sub-unit U_{N}(1) comprises toluene as Z¹_{E,N}(1), wherein more preferably, j=1.
14. The process of embodiment 13, comprising, during a regular operating mode of a sub-unit U_{N}(i), storing a portion of P_{N}(i) in at least one storing device D_{N}(m) as defined in embodiment 5.
15. The process of embodiment 14, comprising starting up a sub-process sp_{N}(i) in a sub-unit U_{N}(i), said starting up comprising feeding at least a portion of P_{N}(i), obtained from said U_{N}(i) during regular operation mode of U_{N}(i), and stored in the at least one storing device D_{N}(m), as S_{N}(i) into U_{N}(i).
16. The process of any one of embodiments 13 to 15, wherein a product composition P_{N}(i), obtained from a sub-unit U_{N}(i), comprises one or more of 2,4-dinitrotoluene as Z¹_{P,N}(i), 2,6-dinitrotoluene as Z²_{P,N}(i), and 2-nitrotoluene as Z³_{P,N}(i).
17. The process of embodiment 16, wherein a product composition P_{N}(i), obtained from a sub-unit U_{N}(i), comprises 2,4-dinitrotoluene as Z¹_{P,N}(i), 2,6-dinitrotoluene as Z²_{P,N}(i), and 2-nitrotoluene as Z³_{P,N}(i).
18. The process of any one of embodiments 13 to 17, further comprising at least one first intermediate treatment sub-process, wherein a first intermediate treatment sub-process comprises, preferably consists of a phase separation sub-process, sp_{S}(i), which is carried out in a sub-unit U_{S}(i), wherein the product composition P_{N}(i) obtained from U_{N}(i), prior to being fed to the sub-unit U_{N}(i+1), is subjected as educt composition E_{S}(i) to separation and is separated during sp_{S}(i) in an organic phase and an aqueous phase, wherein the organic phase, P_{S}(i), is fed as educt composition E_{N}(i+1) into the sub-unit U_{N}(i+1), wherein the sub-unit U_{S}(i) in which sp_{S}(i) is carried out comprises a phase separator.
19. The process of embodiment 18, comprising, during a regular operating mode of a sub-unit U_{S}(i), storing a portion of P_{S}(i) in at least one storing device D_{S}(m) as defined in embodiment 5.
20. The process of embodiment 19, comprising starting up the sub-process sp_{S}(i) in the sub-unit U_{S}(i), said starting up comprising feeding at least a portion of P_{S}(i), obtained from said U_{S}(i) during a regular operating mode of U_{S}(i) and stored in the at least one storing device D_{S}(m), as S_{S}(i) into U_{S}(i).
21. The process of embodiment 19 or 20, comprising starting up the sub-process sp_{N}(i) in a sub-unit U_{N}(i), said starting up comprising feeding at least a portion of P_{S}(i), obtained from said U_{S}(i) and stored in the at least one storing device D_{N}(m), as S_{N}(i) into U_{N}(i).
22. The process of any one of embodiments 18 to 21, wherein a product composition P_{S}(i), obtained from a sub-unit U_{S}(i), comprises one or more of 2,4-dinitrotoluene as Z¹_{P,S}(i), 2,6-dinitrotoluene as Z²_{P,S}(i), and 2-nitrotoluene as Z³_{P,S}(i).
23. The process of any one of embodiments 18 to 22, wherein the product composition P_{S}(i), obtained from the most downstream sub-unit U_{S}(i), comprises one or more of 2,4-dinitrotoluene as Z¹_{P,S}(i) and 2,6-dinitrotoluene as Z²_{P,S}(i), wherein at least 75 mol-%, preferably at least 80 mol-%, more preferably at least 85 mol-%, more preferably at least 90 mol-% of all Z^{k}_{P,S}(i) comprised in the product composition P_{S}(i) obtained from the most downstream sub-unit U_{S}(i) consist of one or more of 2,4-dinitrotoluene as Z¹_{P,S}(i) and 2,6-dinitrotoluene as Z²_{P,S}(i).
24. The process of any one of embodiments 13 to 23, preferably of any one of embodiments 18 to 23, further comprising at least one second intermediate treatment sub-process, wherein a second intermediate treatment sub-process comprises, preferably consists of a washing sub-process, sp_{W}(i), which is carried out in a sub-unit U_{W}(i), wherein a product composition P_{N}(i) obtained from a sub-unit U_{N}(i), or a product composition P_{S}(i) obtained from a sub-unit U_{S}(i), is subjected as educt composition E_{W}(i) to washing, obtaining a product composition P_{W}(i), wherein the sub-unit U_{W}(i) in which sp_{W}(i) is carried out comprises a washing device.
25. The process of embodiment 24, comprising one sub-process sp_{W}(1), wherein sp_{W}(1) is carried out directly downstream of the most downstream sub-process sp_{N}(i), or directly downstream of the most downstream sub-process sp_{S}(i), or directly downstream of the most downstream sub-process sp_{N}(i) and directly downstream of the most downstream sub-process sp_{S}(i), preferably directly downstream of the most downstream sub-process sp_{S}(i).
26. The process of embodiment 24 or 25, comprising, during a regular operating mode of a sub-unit U_{W}(i), storing a portion of P_{W}(i) in at least one storing device D_{W}(m) as defined in embodiment 5.
27. The process of embodiment 26, comprising starting up a sub-process sp_{W}(i) in a sub-unit U_{W}(i), said starting up comprising feeding at least a portion of P_{W}(i), obtained from said U_{W}(i) and stored in the at least one storing device D_{W}(m), as S_{W}(i) into U_{W}(i).
28. The process of any one of embodiments 24 to 27, wherein a product composition P_{W}(i), obtained from a sub-unit U_{W}(i), comprises one or more of 2,4-dinitrotoluene as Z¹_{P,W}(i), 2,6-dinitrotoluene as Z²_{P,W}(i), and 2-nitrotoluene as Z³_{P,W}(i).
29. The process of any one of embodiments 24 to 28, wherein the product composition P_{W}(i), obtained from the most downstream sub-unit U_{W}(i), comprises one or more of 2,4-dinitrotoluene as Z¹_{P,W}(i) and 2,6-dinitrotoluene as Z²_{P,W}(i), wherein at least 75 mol-%, preferably at least 80 mol-%, more preferably at least 85 mol-%, more preferably at least 90 mol-% of all Z^{k}_{P,W}(i) comprised in the product composition P_{W}(i) obtained from the most downstream sub-unit U_{W}(i) consist of one or more of 2,4-dinitrotoluene as Z¹_{P,W}(i) and 2,6-dinitrotoluene as Z²_{P,W}(i).
30. The process of any one of embodiments 1 to 29, preferably of any one of embodiments 13 to 29, more preferably of any one of embodiments 18 to 29, more preferably of any one of embodiments 24 to 29, wherein one or more sub-processes sp(i) comprise a hydrogenation, sp_{H}(i), wherein each sub-unit U_{H}(i) in which a sub-process sp_{H}(i) is carried out comprises a hydrogenation reactor, wherein an educt composition E_{H}(i), being fed into a sub-unit U_{H}(i), comprises at least one hydrogenation agent.
31. The process of embodiment 30, comprising one sub-process sp_{H}(1).
32. The process of embodiment 30 or 31, wherein the at least one aromatic compound Z^{j}_{E.H}(i) comprised in E_{H}(i) is one or more of 2,4-dinitrotoluene as Z¹_{E,H}(i), 2,6-dinitrotoluene as Z²_{E,H}(i), 2-nitrotoluene as Z³_{E,H}(i) and 6-nitrotoluene as Z⁴_{E,H}(i).
33. The process of any one of embodiments 30 to 32, wherein E_{H}(i) comprises one or more of a product composition P_{N}(i) as defined in any one of embodiments 13 to 17, a product composition P_{S}(i) as defined in any one of embodiments 18 to 23, and a product composition P_{W}(i) as defined in any one of embodiments 24 to 29, preferably a product composition P_{W}(i) as defined in any one of embodiments 24 to 28, more preferably the product composition P_{W}(i) as defined in embodiment 29.
34. The process of any one of embodiment 30 to 33, comprising, during a regular operating mode of a sub-unit U_{H}(i), storing a portion of P_{H}(i) in at least one storing device D_{H}(m) as defined in embodiment 5.
35. The process of embodiment 34, comprising starting up a sub-process sp_{H}(i) in a sub-unit U_{H}(i), said starting up comprising feeding at least a portion of P_{H}(i), obtained from said U_{H}(i) and stored in the at least one storing device D_{H}(m), as S_{H}(i) into U_{H}(i).
36. The process of any one of embodiments 30 to 35, wherein a product composition P_{H}(i), obtained from a sub-unit U_{H}(i), comprises one or more of 2,4-diaminotoluene as Z¹_{P,H}(i), 2,6-diaminotoluene as Z²_{P,H}(i), and 2-aminotoluene as Z³_{P,H}(i).
37. The process of embodiment 36, wherein a product composition P_{H}(i), obtained from a sub-unit U_{H}(i), comprises 2,4-diaminotoluene as Z¹_{P,H}(i), 2,6-diaminotoluene as Z²_{P,H}(i), and 2-aminotoluene as Z³_{P,H}(i).
38. The process of any one of embodiments 30 to 37, comprising starting up a sub-process sp_{H}(i) in a sub-unit U_{H}(i), said starting up comprising feeding at least a portion of P_{D}(i), obtained from U_{D}(i) and stored in at least one storing device D_{D,P}(m) as defined in embodiment 41, as S_{H}(i) into U_{H}(i).
39. The process of any one of embodiments 30 to 38, comprising starting up a sub-process sp_{H}(i) in a sub-unit U_{H}(i), said starting up comprising feeding at least a portion of U_{D}(i), obtained from U_{D}(i) and stored in at least one storing device D_{D,C}(m) as defined in embodiment 43, as S_{H}(i) into U_{H}(i).
40. The process of any one of embodiments 30 to 39, further comprising at least one third intermediate treatment sub-process, wherein a third intermediate treatment sub-process comprises, preferably consists of a distillation sub-process, sp_{D}(i), which is carried out in a sub-unit U_{D}(i), wherein preferably, a product composition P_{H}(i) obtained from a sub-unit U_{H}(i) is subjected as educt composition E_{D}(i) to distillation, obtaining a product composition P_{D}(i), wherein the sub-unit U_{D}(i) in which sp_{D}(i) is carried out comprises a distillation device and wherein in U_{D}(i), E_{D}(i) is separated in P_{D}(i) and at least one further distillation composition C_{D}(i).
41. The process of embodiment 40, comprising, during a regular operating mode of a sub-unit U_{D}(i), storing a portion of P_{D}(i) in at least one storing device D_{D,P}(m) as defined in embodiment 5.
42. The process of embodiment 41, comprising starting up a sub-process sp_{D}(i) in a sub-unit U_{D}(i), said starting up comprising feeding at least a portion of P_{D}(i), obtained from said U_{D}(i) and stored in the at least one storing device D_{D,P}(m), as S_{D}(i) into U_{D}(i).
43. The process of any one of embodiments 38 to 42, comprising, during a regular operating mode of a sub-unit U_{D}(i), storing a portion of C_{D}(i) in at least one storing device D_{D,C}(m) as defined in embodiment 5.
44. The process of embodiment 43, comprising starting up a sub-process sp_{D}(i) in a sub-unit U_{D}(i), said starting up comprising feeding at least a portion of C_{D}(i), obtained from said U_{D}(i) and stored in the at least one storing device D_{D,C}(m), as S_{D}(i) into U_{D}(i).
45. The process of any one of embodiments 38 to 44, wherein a product composition P_{D}(i), obtained from a sub-unit U_{D}(i), comprises one or more of 2,4-diaminotoluene as Z¹_{P,D}(i), 2,6-diaminotoluene as Z²_{P,D}(i), and 2-aminotoluene as Z³_{P,D}(i).
46. The process of embodiment 45, wherein a product composition P_{D}(i), obtained from a sub-unit U_{D}(i), comprises 2,4-diaminotoluene as Z¹_{P,D}(i), 2,6-diaminotoluene as Z²_{P,D}(i), and 2-aminotoluene as Z³_{P,D}(i).
47. The process of any one of embodiments 40 to 46, the weight ratio of the total weight of 2,4-diaminotoluene as Z¹_{P,D}(i), 2,6-diaminotoluene as Z²_{P,D}(i), and 2-aminotoluene as Z³_{P,D}(i) comprised in P_{D}(i) relative to the total weight of 2,4-diaminotoluene as Z¹_{P,H}(i), 2,6-diaminotoluene as Z²_{P,H}(i), and 2-aminotoluene as Z³_{P,H}(i) comprised in P_{H}(i) is greater than 1:1, more preferably in the range of from 1.05:1 to 2.5:1, more preferably in the range of from 1.3:1 to 2.2:1, more preferably in the range of from 1.5:1 to 2.1:1.
48. The process of any one of embodiments 40 to 46, wherein the weight ratio of the product composition P_{D}(i) relative to the at least one further distillation composition C_{D}(i) is in the range of from 25:1 to 2:1, more preferably in the range of from 20:1 to 5:1.
49. The process of any one of embodiments 38 to 48, comprising starting up a sub-process sp_{D}(i) in a sub-unit U_{D}(i), said starting up comprising feeding at least a portion of P_{H}(i), obtained from U_{H}(i) and stored in the at least one storing device D_{H}(m) as defined in embodiment 35, as S_{D}(i) into U_{D}(i).
50. The process of any one of embodiments 13 to 49, preferably of any one of embodiments 18 to 47, more preferably of any one of embodiments 24 to 49, more preferably of any one of embodiments 30 to 47, more preferably of any one of embodiments 40 to 49, wherein one or more sub-processes sp(i) comprise a phosgenation, sp_{P}(i), wherein each sub-unit U_{P}(i) in which a sub-process sp_{P}(i) is carried out comprises a phosgenation reactor, wherein an educt composition E_{P}(i), being fed into a sub-unit U_{P}(i), comprises at least one phosgenation agent.
51. The process of embodiment 50, comprising one sub-process sp_{P}(1).
52. The process of embodiment 50 or 51, wherein the at least one aromatic compound Z^{j}_{E.P}(i) comprised in E_{P}(i) is one or more of 2,4-diaminotoluene as Z¹_{E,P}(i), 2,6-diaminotoluene as Z²_{E,P}(i), 2-aminotoluene as Z³_{E,P}(i) and 6-aminotoluene as Z⁴_{E,P}(i).
53. The process of any one of embodiments 50 to 52, wherein E_{P}(i) comprises one or more of a product composition P_{H}(i) as defined in embodiment 36 or 37, a product composition P_{D}(i) as defined in any one of embodiments 45 to 48, and a distillation composition C_{D}(i) as defined in embodiment 40, preferably a product composition P_{D}(i) as defined in any one of embodiments 45 to 48, more preferably the product composition P_{D}(i) as defined in any one of embodiments 46 to 48.
54. The process of any one of embodiment 50 to 53, comprising, during a regular operating mode of a sub-unit U_{P}(i), storing a portion of Pₚ(i) in at least one storing device Dₚ(m) as defined in embodiment 5.
55. The process of embodiment 54, comprising starting up a sub-process sp_{P}(i) in a sub-unit U_{P}(i), said starting up comprising feeding at least a portion of Pₚ(i), obtained from said U_{P}(i) and stored in the at least one storing device Dₚ(m), as Sₚ(i) into U_{P}(i).
56. The process of any one of embodiments 50 to 55, wherein a product composition Pₚ(i), obtained from a sub-unit U_{P}(i), comprises one or more of 2,4-toluene diisocyanate as Z¹_{P,P}(i), 2,6-toluene diisocyanate as Z²_{P,P}(i), and 2-toluene isocyanate as Z³_{P,P}(i).
57. The process of embodiment 56, wherein a product composition Pₚ(i), obtained from a sub-unit U_{P}(i), comprises 2,4-toluene diisocyanate as Z¹_{P,P}(i), 2,6-toluene diisocyanate as Z²_{P,P}(i), and 2-toluene isocyanate as Z³_{P,P}(i).
58. The process of any one of embodiments 1 to 57, wherein the process is a continuous or discontinuous process, preferably a continuous process.
59. A chemical unit U for carrying out the process according to any one of embodiments 1 to 58.
60. The chemical unit of embodiment 59, comprising
(i.1) one or more sub-units U_{N}(i), preferably two sub-units U_{N}(1) and U_{N}(2);
(i.2) one or more sub-units U_{S}(i), preferably two sub-units U_{S}(1) and U_{S}(²);
(i.3) one or more sub-units U_{W}(i), preferably one sub-unit U_{W}(1);
(i.4) one or more storing devices D_{N}(m), preferably two storing devices D_{N}(1) and D_{N}(2);
wherein U_{N}(1) is arranged upstream of U_{S}(1), U_{S}(1) is arranged upstream U_{N}(2), U_{N}(2) is arranged upstream U_{S}(2), and U_{S}(2) is arranged upstream of U_{W}(1);
(ii.1) one or more sub-units U_{H}(i), preferably one sub-unit U_{H}(1);
(ii.2) one or more sub-units U_{D}(i), preferably one sub-unit U_{D}(1);
(ii.3) one or more storing devices D_{H}(m), preferably one storing device D_{H}(1);
(ii.4) one or more storing devices D_{D}(m), preferably two storing devices D_{D,P}(1) and D_{D,C}(1);
wherein U_{H}(1) is arranged downstream of U_{W}(1) and upstream U_{D}(1);
(iii.1) one or more sub-units U_{P}(i), preferably one sub-unit U_{P}(1);
wherein U_{P}(1) is arranged downstream of U_{D}(1).

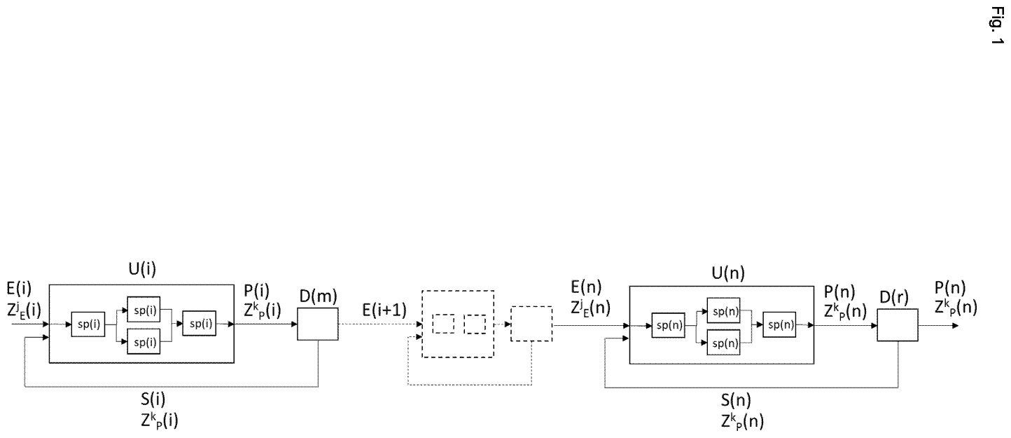

FIG. 1 shows a flow diagram according to the process of the present invention. Depicted is a process comprising n chemical sub-processes sp(i). The sub-process sp(i) is carried out in a chemical processing sub-unit U(i) where i=1 . . . n, with n≥1.

For example, when n=1, during regular operation mode of a sub-unit U_{N}(1) for nitration, the sub-process sp_{N}(1) being carried out in said sub-unit U_{N}(1) comprises feeding an educt composition E_{N}(1) comprising at least one aromatic compound Z^{j}_{E}(1), such as toluene, into said sub-unit U_{N}(1). The composition E_{N}(1) comprising at least one aromatic compound Z^{j}_{E}(1) is processed in said sub-unit to obtain a product composition P_{N}(1) comprising at least one aromatic compound Z^{k}ₚ(1), k≥1 being dinitrotoluene.

For example, when n=2, for nitration, two sub-units U_{N}(1) and U_{N}(2), the sub-units U_{N}(1) and U_{N}(2), are part of a chemical unit U, wherein U further comprises, in addition to the 2 chemical processing sub-units U_{N}(1) and U_{N}(2), one storing device D_{N}(1). During regular operation mode of the sub-unit U_{N}(1), a portion of the product P_{N}(1) obtained from U_{N}(1) and comprising dinitrotoluene is stored in a storing device D_{N}(1), and the remaining portion of the product P_{N}(1) is removed from U_{N}(1) and fed to either U_{N}(2) or to a further downstream sub-unit. Sub-unit U_{N}(1) is shut down for maintenance and regular operation is stopped. After a maintenance routine of U_{N}(1) is completed, in the starting up process of the sub-process Sp_{N}(1) in sub-unit U_{N}(1), a portion of P_{N}(1) (dinitrotoluene), obtained from said U_{N}(1) during regular operation mode of U_{N}(1) and U_{N}(2), and stored in the storing device D_{N}(1), is fed back into the nitration sub-unit U_{N}(1) as a start-up composition S_{N}(1). Accordingly, the start-up composition S_{N}(1)≠educt composition E_{N}(1). Depicted as well are optional intermediate processes and units symbolized as a broken lines between the two sub-units.

### Cited Literature

WO 2015/197522 A1
WO 2017/050776 A1

1.-17. (canceled)

18. A chemical process for preparing at least one aromatic isocyanate, said process comprising n chemical sub-processes sp(i), wherein a sub-process sp(i) is carried out in a chemical processing sub-unit U(i), i=1 . . . n, n≥1,
wherein during a regular operating mode of a sub-unit U(i), the sub-process sp(i) being carried out in said sub-unit U(i) comprises feeding an educt composition E(i) comprising at least one aromatic compound Z^{j}_{E}(i), j≥1, into said sub-unit U(i), processing said composition E(i) in said sub-unit U(i) and obtaining a product composition P(i) comprising at least one aromatic compound Z^{k}ₚ(i), k≥1,
wherein the chemical process comprises starting up said sub-process sp(i) in said sub-unit U(i), wherein said starting-up comprises processing a chemical start-up composition S(i) in said sub-unit U(i), wherein said S(i)≠E(i) and wherein said S(i) comprises at least one of the aromatic compounds Z^{k}ₚ(i).

19. The process of claim 18, wherein, if n≥2, at least 2, wherein U further comprises, in addition to the n chemical processing sub-units U(i), r storing devices D(m), m=1 . . . r, r≥1; said chemical process further comprising, during a regular operating mode of a sub-unit U(i), removing the product composition P(i) from said sub-unit U(i) and transferring a portion of said product composition P(i) removed from said sub-unit U(i) into a storing device D(m); said chemical process further comprising, during starting up the sub-process sp(i), feeding at least a portion of P(i), stored in D(m) during the regular operating mode of the sub-unit U(i), as at least a portion of the chemical composition S(i) into the sub-unit U(i).

20. The process of claim 18, wherein the sub-unit U(i) is selected from the group consisting of a chemical reactor, a distillation column, a rectification column, a tank, a phase separator, a washing device, and a combination of two or more thereof, wherein at least one sub-process sp(i) comprises a nitration and at least one further sub-process sp(i) comprises a hydrogenation.

21. The process of claim 18, wherein one or more sub-processes sp(i) comprise a nitration, sp_{N}(i), wherein each sub-unit U_{N}(i) in which a sub-process sp_{N}(i) is carried out comprises a nitration reactor, wherein an educt composition E_{N}(i), being fed into a sub-unit U_{N}(i), comprises at least one nitrating agent;
wherein said process comprises two or more sub-processes sp_{N}(i) which are carried out in sequence, wherein at last a portion of the product composition P_{N}(i) obtained from a sub-unit U_{N}(i) is fed, optionally after an intermediate treatment sub-process, as educt composition E_{N}(i+1) into the sub-unit U_{N}(i+1) downstream of U_{N}(i) during a regular operating mode of U_{N}(i+1);
wherein the educt composition E_{N}(1) being fed into the first, most upstream sub-unit U_{N}(1) comprises toluene as Z¹_{E,N}(1);
wherein said chemical process comprises, during a regular operating mode of a sub-unit U_{N}(i), storing a portion of P_{N}(i) in at least one storing device D_{N}(m), m=1 . . . r, r≥1;
wherein said chemical process comprises, starting up a sub-process sp_{N}(i) in a sub-unit U_{N}(i), said starting up comprising feeding at least a portion of P_{N}(i), obtained from said U_{N}(i) during regular operation mode of U_{N}(i), and stored in the at least one storing device D_{N}(m), as S_{N}(i) into U_{N}(i).

22. The process of claim 21, wherein a product composition P_{N}(i), obtained from a sub-unit U_{N}(i), comprises one or more of 2,4-dinitrotoluene as Z¹_{P,N}(i), 2,6-dinitrotoluene as Z²_{P,N}(i), and 2-nitrotoluene as Z³_{P,N}(i).

23. The process of claim 21, further comprising at least one first intermediate treatment sub-process, wherein a first intermediate treatment sub-process comprises a phase separation sub-process, sp_{S}(i), which is carried out in a sub-unit U_{S}(i), wherein the product composition P_{N}(i) obtained from U_{N}(i), prior to being fed to the sub-unit U_{N}(i+1), is subjected as educt composition E_{S}(i) to separation and is separated during sp_{S}(i) in an organic phase and an aqueous phase, wherein the organic phase, P_{S}(i), is fed as educt composition E_{N}(i+1) into the sub-unit U_{N}(i+1), wherein the sub-unit U_{S}(i) in which sp_{S}(i) is carried out comprises a phase separator;
wherein said chemical comprises, during a regular operating mode of a sub-unit U_{S}(i), storing a portion of P_{S}(i) in at least one storing device D_{S}(m), m=1 . . . r, r≥1;
wherein said chemical comprises starting up the sub-process sp_{S}(i) in the sub-unit U_{S}(i), said starting up comprising feeding at least a portion of P_{S}(i), obtained from said U_{S}(i) during a regular operating mode of U_{S}(i) and stored in the at least one storing device D_{S}(m), as S_{S}(i) into U_{S}(i);

24. The process of claim 23, comprising starting up the sub-process sp_{N}(i) in a sub-unit U_{N}(i), said starting up comprising feeding at least a portion of P_{S}(i), obtained from said U_{S}(i) and stored in the at least one storing device D_{N}(m), as S_{N}(i) into U_{N}(i).

25. The process of claim 23, wherein a product composition P_{S}(i), obtained from a sub-unit U_{S}(i), comprises one or more of 2,4-dinitrotoluene as Z¹_{P,S}(i), 2,6-dinitrotoluene as Z²_{P,S}(i), and 2-nitrotoluene as Z³_{P,S}(i);
wherein at least 75 mol-% comprised in the product composition P_{S}(i) obtained from the most downstream sub-unit U_{S}(i) consist of one or more of 2,4-dinitrotoluene as Z¹_{P,S}(i) and 2,6-dinitrotoluene as Z²_{P,S}(i).

26. The process of claim 23, further comprising at least one second intermediate treatment sub-process, wherein a second intermediate treatment sub-process comprises a washing sub-process, sp_{W}(i), which is carried out in a sub-unit U_{W}(i), wherein a product composition P_{N}(i) obtained from a sub-unit U_{N}(i), or a product composition P_{S}(i) obtained from a sub-unit U_{S}(i), is subjected as educt composition E_{W}(i) to washing, obtaining a product composition P_{W}(i), wherein the sub-unit U_{W}(i) in which sp_{W}(i) is carried out comprises a washing device;
wherein said process comprises one sub-process sp_{W}(1), wherein sp_{W}(1) is carried out directly downstream of the most downstream sub-process sp_{N}(i), or directly downstream of the most downstream sub-process sp_{S}(i), or directly downstream of the most downstream sub-process sp_{N}(i) and directly downstream of the most downstream sub-process sp_{S}(i);
wherein said process comprises, during a regular operating mode of a sub-unit U_{W}(i), storing a portion of P_{W}(i) in at least one storing device D_{W}(m), m=1 . . . r, r≥1;
wherein said process comprises starting up a sub-process sp_{W}(i) in a sub-unit U_{W}(i), said starting up comprising feeding at least a portion of P_{W}(i), obtained from said U_{W}(i) and stored in the at least one storing device D_{W}(m), as S_{W}(i) into U_{W}(i).

27. The process of claim 26, wherein a product composition P_{W}(i), obtained from a sub-unit U_{W}(i), comprises one or more of 2,4-dinitrotoluene as Z¹_{P,W}(i), 2,6-dinitrotoluene as Z²_{P,W}(i), and 2-nitrotoluene as Z³_{P,W}(i);
wherein at least 75 mol-% comprised in the product composition P_{W}(i) obtained from the most downstream sub-unit U_{W}(i) consist of one or more of 2,4-dinitrotoluene as Z¹_{P,W}(i) and 2,6-dinitrotoluene as Z²_{P,W}(i).

28. The process of claim 18, wherein one or more sub-processes sp(i) comprise a hydrogenation, sp_{H}(i), wherein each sub-unit U_{H}(i) in which a sub-process sp_{H}(i) is carried out comprises a hydrogenation reactor, wherein an educt composition E_{H}(i), being fed into a sub-unit U_{H}(i), comprises at least one hydrogenation agent;
wherein the at least one aromatic compound Z^{j}_{E.H}(i) comprised in E_{H}(i) is one or more of 2,4-dinitrotoluene as Z¹_{E,H}(i), 2,6-dinitrotoluene as Z²_{E,H}(i), 2-nitrotoluene as Z³_{E,H}(i) and 6-nitrotoluene as Z⁴_{E,H}(i);
wherein said process comprises, during a regular operating mode of a sub-unit U_{H}(i), storing a portion of P_{H}(i) in at least one storing device D_{H}(m);
wherein said process comprises starting up a sub-process sp_{H}(i) in a sub-unit U_{H}(i), said starting up comprising feeding at least a portion of P_{H}(i), obtained from said U_{H}(i) and stored in the at least one storing device D_{H}(m), as S_{H}(i) into U_{H}(i).

29. The process of claim 28, wherein a product composition P_{H}(i), obtained from a sub-unit U_{H}(i), comprises one or more of 2,4-diaminotoluene as Z¹_{P,H}(i), 2,6-diaminotoluene as Z²_{P,H}(i), and 2-aminotoluene as Z³_{P,H}(i).

30. The process of claim 26, further comprising at least one third intermediate treatment sub-process, wherein a third intermediate treatment sub-process comprises a distillation sub-process, sp_{D}(i), which is carried out in a sub-unit U_{D}(i), wherein a product composition P_{H}(i) obtained from a sub-unit U_{H}(i) is subjected as educt composition E_{D}(i) to distillation, obtaining a product composition P_{D}(i), wherein the sub-unit U_{D}(i) in which sp_{D}(i) is carried out comprises a distillation device and wherein in U_{D}(i), E_{D}(i) is separated in P_{D}(i) and at least one further distillation composition C_{D}(i);
wherein
said process comprises, during a regular operating mode of a sub-unit U_{D}(i), storing a portion of P_{D}(i) in at least one storing device D_{D,P}(m), m=1 . . . r, r≥1;
said process comprising starting up a sub-process sp_{D}(i) in a sub-unit U_{D}(i), said starting up comprising feeding at least a portion of P_{D}(i), obtained from said U_{D}(i) and stored in the at least one storing device D_{D,P}(m), as S_{D}(i) into U_{D}(i);
and/or
said process comprises, during a regular operating mode of a sub-unit U_{D}(i), storing a portion of C_{D}(i) in at least one storing device D_{D,C}(m);
said process comprising starting up a sub-process sp_{D}(i) in a sub-unit U_{D}(i), said starting up comprising feeding at least a portion of C_{D}(i), obtained from said U_{D}(i) and stored in the at least one storing device D_{D,C}(m), as S_{D}(i) into U_{D}(i).

31. The process of claim 30, comprising
starting up a sub-process sp_{H}(i) in a sub-unit U_{H}(i), said starting up comprising feeding at least a portion of P_{D}(i), obtained from U_{D}(i) and stored in at least one storing device D_{D,P}(m), m=1 . . . r r≥1, as S_{H}(i) into U_{H}(i);
and/or
starting up a sub-process sp_{H}(i) in a sub-unit U_{H}(i), said starting up comprising feeding at least a portion of U_{D}(i), obtained from U_{D}(i) and stored in at least one storing device D_{D},c(m), m=1 . . . r, r≥1, as S_{H}(i) into U_{H}(i).

32. The process of claim 30, wherein a product composition P_{D}(i), obtained from a sub-unit U_{D}(i), comprises one or more of 2,4-diaminotoluene as Z¹_{P,D}(i), 2,6-diaminotoluene as Z²_{P,D}(i), and 2-aminotoluene as Z³_{P,D}(i).

33. The process of claim 30, comprising starting up a sub-process sp_{D}(i) in a sub-unit U_{D}(i), said starting up comprising feeding at least a portion of P_{H}(i), obtained from U_{H}(i) and stored in the at least one storing device D_{H}(m), as S_{D}(i) into U_{D}(i).

34. A chemical unit U for carrying out the process according to claim 18, wherein said chemical unit U comprises
(i.1) one or more sub-units U_{N}(i);
(i.2) one or more sub-units U_{S}(i);
(i.3) one or more sub-units U_{W}(i);
(i.4) one or more storing devices D_{N}(m);
wherein U_{N}(l) is arranged upstream of U_{S}(1), U_{S}(1) is arranged upstream U_{N}(2), U_{N}(2) is arranged upstream U_{S}(2), and U_{S}(2) is arranged upstream of U_{W}(1);
(ii.1) one or more sub-units U_{H}(i);
(ii.2) one or more sub-units U_{D}(i);
(ii.3) one or more storing devices D_{H}(m);
(ii.4) one or more storing devices D_{D}(m);
wherein U_{H}(l) is arranged downstream of U_{W}(1) and upstream U_{D}(l);
(iii.1) one or more sub-units U_{P}(i);
wherein U_{P}(1) is arranged downstream of U_{D}(1).
